(19) United States
(12) Patent Application Publication
(10) Pub. No.: US 2026/0157611 A1
(43) Pub. Date: Jun. 11, 2026

(54) METHODS AND SYSTEMS FOR MEDICAL IMAGING WITH MULTI-MODAL ADAPTOR COUPLING

(71) Applicant: ACTIV Surgical, Inc., Boston, MA (US)

(72) Inventors: Allen Bates, Boston, MA (US); Charlie Beurskens, Boston, MA (US); Anderson Mach, Boston, MA (US); Stephen Tully, Boston, MA (US)

(21) Appl. No.: 18/858,171

(22) PCT Filed: Apr. 21, 2023
(86) PCT No.: PCT/US2023/019457

§ 371 Date: Oct. 18, 2024

Related U.S. Application Data
(60) Provisional application No. 63/333,764, filed on Apr. 22, 2022.

(51) Int. Cl. A61B 1/00; A61B 1/04

(52) U.S. Cl. CPC A61B 1/00188; A61B 1/046

(57) ABSTRACT

The present disclosure provides a system for medical imaging. The system may comprise an optical adapter for visualizing a surgical scene using visible light and non-visible light. The optical adapter may be attachable to a scope and an imaging device. The optical adapter may comprise an optics assembly and an imaging sensor configured for imaging of the surgical scene.

### CROSS-REFERENCE

This application claims the benefit of U.S. Provisional Application No. 63/333,764, filed Apr. 22, 2022, which application is incorporated herein by reference in its entirety.

### BACKGROUND

Medical imaging technology (e.g., a scope assembly, such as an endoscope) may be used to capture images or video data of internal anatomical features of a subject or patient during medical or surgical procedures. The images or video data captured may be processed and manipulated to provide medical practitioners (e.g., surgeons, medical operators, technicians, etc.) with a visualization of internal structures or processes within a patient or subject.

### SUMMARY

Recognized herein are various limitations with optical adapter systems currently available, many of which rely on complex focusing optics to align and/or focus multiple images obtained using different camera systems. The present application relates generally to optical systems and, more particularly, to optical adapters that are capable of imaging in multiple modalities, and universally compatible with any type of imaging device, regardless of hardware configuration and/or form factor.

In an aspect, the present disclosure provides a system. The system may comprise: an optical adapter for visualizing an image of tissue using visible light and non-visible light, wherein the optical adapter is attachable to a scope and an imaging device, and wherein the optical adapter comprises: an optics assembly for directing (i) the visible light to the imaging device and (ii) the non-visible light to an imaging sensor, wherein the visible light exiting the optics assembly toward the imaging device comprises about the same image size, image collimation, and image orientation as the visible light entering the optics assembly from the scope.

In some embodiments, the imaging device comprises a C-mount imaging device or an imaging device with an integrated coupler. In some embodiments, the optics assembly comprises a first lens assembly, a second lens assembly, and a prism system between the first lens assembly and the second lens assembly.

In another aspect, the present disclosure provides a system. The system my comprise: an optical adapter for visualizing an image of tissue using visible light and non-visible light, wherein the optical adapter is attachable to a scope and an imaging device, and wherein the optical adapter comprises: an optics assembly for directing (i) the visible light to the imaging device and (ii) the non-visible light to an imaging sensor, wherein the imaging device comprises a C-mount imaging device or an imaging device with an integrated coupler, and wherein the visible light directed to the imaging device comprises about the same image size, image collimation, and image orientation when using either the C-mount imaging device or the imaging device with the integrated coupler.

In some embodiments, the visible light exiting the optics assembly toward the imaging device comprises about the same image size, image collimation, and image orientation as the visible light entering the optics assembly from the scope. In some embodiments, the optics assembly comprises a first lens assembly, a second lens assembly, and a prism system between the first lens assembly and the second lens assembly.

In another aspect, the present disclosure provides a system. The system may comprise: an optical adapter for visualizing an image of tissue using visible light and non-visible light, wherein the optical adapter is attachable to a scope and an imaging device, and wherein the optical adapter comprises: an optics assembly for directing (i) the visible light to the imaging device and (ii) the non-visible light to an imaging sensor, wherein the optics assembly comprises a first lens assembly, a second lens assembly, and a prism system between the first lens assembly and the second lens assembly.

In some embodiments, the imaging device comprises a C-mount imaging device or an imaging device with an integrated coupler, and wherein the visible light directed to the imaging device comprises about the same image size, image collimation, and image orientation when using either the C-mount imaging device or the imaging device with the integrated coupler. In some embodiments, the visible light exiting the optics assembly toward the imaging device comprises about the same image size, image collimation, and image orientation as the visible light entering the optics assembly from the scope.

In some embodiments, the imaging device comprises the C-mount imaging device and a coupler for the C-mount imaging device. In some embodiments, the coupler is a native coupler for the C-mount imaging device.

In some embodiments, the prism system comprises a pechan prism pair, a porro prism pair, an Uppendahl prism system, Abbe-Porro prism system, or an Abbe-Koenig prism system.

In some embodiments, the optics assembly comprises a first optic, a second optic, and third optic, wherein the second optic is positioned between the first optic and the third optic. In some embodiments, the first optic is configured to direct at least a portion of the visible light to the second optic, and wherein the second optic is configured to manipulate the visible light to adjust a property or a characteristic of an image associated with or derivable from the visible light. In some embodiments, the third optic is configured to receive the visible light from the second optic and provide substantially parallel beams of the visible light directly to the imaging device, wherein the substantially parallel beams of the visible light substantially replicate an image signal that is received by or from the scope.

In some embodiments, a distance between the scope and the imaging device is less than six inches. In some embodiments, the optics assembly comprises an optical element configured to direct the visible light to the imaging device and the non-visible light to the imaging sensor. In some embodiments, the optical element comprises a beam splitter. In some embodiments, the optical element comprises a dichroic mirror or lens. In some embodiments, the optical element is positioned upstream of the first optic or the first lens assembly. In some embodiments, the optical element is positioned between the first optic and the third optic or between the first lens assembly and the second lens assembly. In some embodiments, the optical element is positioned downstream of the third optic or the second lens assembly.

In some embodiments, the system further comprises the imaging device, wherein the imaging device is integrated with the system. In some embodiments, the optics assembly comprises at least one achromatic lens to reduce spherical and chromatic aberrations induced or created by the at least one prism. In some embodiments, the at least one achromatic lens comprises an achromatic singlet lens or an achromatic doublet lens.

In some embodiments, the optics assembly is configured to remove or reduce aberrations in one or more output images generated using the parallel beams of the visible light. In some embodiments, one or more lenses of the optics assembly comprises a telecentric lens. In some embodiments, the optics assembly is configured to provide a telecentric pupil space.

In some embodiments, the optics assembly is optically symmetric relative to the at least one prism to remove or reduce odd order aberrations. In some embodiments, the optics assembly comprises at least one prism, wherein the at least one prism is displaced to compensate for a shift in an optical path or axis of the visible light that is induced or caused by one or more components or sub-components of the optics assembly. In some embodiments, the optics assembly is configured to eliminate or prevent a formation, projection, or placement of one or more intermediate image planes on a portion, surface, or edge of the at least one prism. In some embodiments, the optics assembly is configured to provide an output signal that substantially maintains a quality of the image signal received by or from the scope.

In some embodiments, the optical adapter comprises a sealed housing comprising one or more windows for receiving an input optical beam comprising the visible light and/or the non-visible light. In some embodiments, the optical adapter comprises a channel for confining or controlling a divergence of the input optical beam. In some embodiments, the channel comprises a high refractive index material. In some embodiments, the optical adapter is attachable to focusing optics integrated with the imaging device.

In some embodiments, the optics assembly is configured to diverge light received by the optical adapter into a plurality of paths based on wavelength. In some embodiments, the optics assembly is configured to separate wavelengths of light without distorting the image signal received by or from the scope. In some embodiments, the optical adapter is configured to pass white light through the optics assembly to the imaging device. In some embodiments, the optics assembly is configured to flip or rotate an RGB image derivable from the visible light in order to replicate the image signal received by or from the scope. In some embodiments, the optics assembly is configured to provide or maintain a constant optical axis for one or more optical signals received by the optical adapter and/or transmitted to the imaging device. In some embodiments, the constant optical axis extends from a first end of the optical adapter to a second end of the optical adapter, wherein the imaging device is positioned at the second end of the optical adapter. In some embodiments, the optics assembly is configured to actively or passively separate light based on wavelength.

In some embodiments, the optical adapter is configured for multiple uses. In some embodiments, the optical adapter is configured for single use.

In some embodiments, wherein the system further comprises an alignment system to adjust (i) an alignment of the imaging device relative to the imaging sensor or (ii) an alignment of the imaging sensor relative to the imaging device. In some embodiments, the alignment system is configured to calibrate the imaging sensor relative to the imaging device. In some embodiments, the system further comprises one or more sensors configured to provide feedback on (i) an alignment of the imaging device relative to the imaging sensor or (ii) an alignment of the imaging sensor relative to the imaging device. In some embodiments, the system further comprises an alignment system configured to automatically adjust (i) the alignment of the imaging device relative to the imaging sensor or (ii) the alignment of the imaging sensor relative to the imaging device, based on one or more measurements obtained using the one or more sensors.

In some embodiments, the optical adapter further comprises a connection interface integrated with a housing of the optical adapter, wherein the connection interface is configured to releasably couple the optical adapter to an eyepiece of (i) the imaging device and/or (ii) focusing optics integrated with the imaging device. In some embodiments, the imaging device comprises the human eye. In some embodiments, the optics assembly comprises at least one achromatic doublet combined with a singlet to reduce spherical and chromatic aberrations induced or created by a prism within the optics assembly. In some embodiments, the imaging sensor is configured for laser speckle imaging of the surgical scene.

In another aspect, the present disclosure provides a system. The system may comprise: an optical adapter for visualizing an image of tissue using visible light and non-visible light, wherein the optical adapter is attachable to a scope and an imaging device, and wherein the optical adapter comprises: an optics assembly for directing (i) the visible light to the imaging device and (ii) the non-visible light to an imaging sensor, wherein the optics assembly comprises a first optic, a second optic, and third optic, wherein the second optic is positioned between the first optic and the third optic, wherein the first optic is configured to direct at least a portion of the visible light to the second optic, wherein the second optic is configured to manipulate the visible light to adjust a property or a characteristic of an image associated with or derivable from the visible light, and wherein the third optic is configured to receive the visible light from the second optic and provide substantially parallel beams of the visible light directly to the imaging device, wherein the substantially parallel beams of the visible light substantially replicate an image signal that is received by or from the scope.

In some embodiments, the optics assembly comprises an optical element configured to direct the visible light to the imaging device and the non-visible light to the imaging sensor. In some embodiments, the optical element comprises a beam splitter. In some embodiments, the optical element comprises a dichroic mirror or lens. In some embodiments, the optical element is positioned upstream of the first optic. In some embodiments, the optical element is positioned between the first optic and the third optic. In some embodiments, the optical element is positioned downstream of the third optic.

In some embodiments, the first optic is configured to receive non-parallel beams of the visible light from the scope and transmit the non-parallel beams of the visible light to the second optic. In some embodiments, the parallel beams of the visible light are usable to generate an output image having a same property or characteristic as a reference image associated with the image signal received by or from the scope. In some embodiments, the property or characteristic comprises an image orientation, an image quality, or an image fidelity. In some embodiments, the image signal is replicated without post-processing of the output image. In some embodiments, the parallel beams of the visible light form a nominally collimated beam. In some embodiments, the nominally collimated beam is usable to generate an RGB or visible light image of the surgical scene that is not inverted, rotated, or visually distorted relative to the surgical scene as viewed from or through the scope. In some embodiments, the parallel beams of the visible light are focused on a plurality of different regions of a light sensing unit of the imaging device.

In some embodiments, the optics assembly is configured to manipulate the image associated with or derivable from the visible light in order to replicate the image signal that is received by or from the scope. In some embodiments, the optics assembly is configured to manipulate the image associated with or derivable from the visible light by rotating, reorienting, flipping, mirroring, inverting, or resizing the image. In some embodiments, the optics assembly comprises one or more lenses and at least one prism. In some embodiments, the first optic comprises a first lens or a first lens assembly comprising the first lens, the second optic comprises the at least one prism, and the third optic comprises a second lens or a second lens assembly comprising the second lens. In some embodiments, the first lens or lens assembly is configured to produce the image associated with or derivable from the visible light inside or within the at least one prism. In some embodiments, the at least one prism is configured to manipulate the image associated with or derivable from the visible light received from the first lens or lens assembly in order to replicate the image signal received by or from the scope. In some embodiments, the second lens or lens assembly is configured to receive the manipulated visible light beams from the at least one prism and to direct the parallel beams of the visible light to the imaging device, wherein the parallel beams of the visible light correspond to the image manipulated by the at least one prism.

In some embodiments, the at least one prism comprises a roof prism. In some embodiments, the at least one prism comprises a Porro prism. In some embodiments, the at least one prism is configured to fold an optical path of the visible light. In some embodiments, at least one of the first lens or lens assembly and the second lens or lens assembly comprises a symmetrical lens. In some embodiments, the first lens or lens assembly and the second lens or lens assembly are provided in a symmetrical configuration relative to the at least one prism. In some embodiments, the optics assembly comprises at least one achromatic lens to reduce spherical and chromatic aberrations induced or created by the at least one prism. In some embodiments, the at least one achromatic lens comprises an achromatic singlet lens or an achromatic doublet lens.

In some embodiments, the optics assembly is configured to remove or reduce aberrations in one or more output images generated using the parallel beams of the visible light. In some embodiments, the one or more lenses comprise a telecentric lens. In some embodiments, the optics assembly is configured to provide a telecentric pupil space. In some embodiments, the optics assembly is optically symmetric relative to the at least one prism to remove or reduce odd order aberrations. In some embodiments, the at least one prism is displaced to compensate for a shift in an optical path or axis of the visible light that is induced or caused by one or more components or sub-components of the optics assembly. In some embodiments, the optics assembly is configured to eliminate or prevent a formation, projection, or placement of one or more intermediate image planes on a portion, surface, or edge of the at least one prism.

In some embodiments, the optics assembly is configured to provide an output signal that substantially maintains a quality of the image signal received by or from the scope. In some embodiments, the optical adapter comprises a sealed housing comprising one or more windows for receiving an input optical beam comprising the visible light and/or the non-visible light. In some embodiments, the optical adapter comprises a channel for confining or controlling a divergence of the input optical beam. In some embodiments, the channel comprises a high refractive index material.

In some embodiments, the optical adapter is attachable to focusing optics integrated with the imaging device. In some embodiments, the optics assembly is configured to diverge light received by the optical adapter into a plurality of paths based on wavelength. In some embodiments, the optics assembly is configured to separate wavelengths of light without distorting the image signal received by or from the scope. In some embodiments, the optical adapter is configured to pass white light through the optics assembly to the imaging device. In some embodiments, the optics assembly is configured to flip or rotate an RGB image derivable from the visible light in order to replicate the image signal received by or from the scope.

In some embodiments, the optics assembly is configured to provide or maintain a constant optical axis for one or more optical signals received by the optical adapter and/or transmitted to the imaging device. In some embodiments, the constant optical axis extends from a first end of the optical adapter to a second end of the optical adapter, wherein the imaging device is positioned at the second end of the optical adapter. In some embodiments, the optics assembly is configured to actively or passively separate light based on wavelength. In some embodiments, the optical adapter is configured for multiple uses. In some embodiments, the optical adapter is configured for single use.

In some embodiments, the system further comprises an alignment system to adjust (i) an alignment of the imaging device relative to the imaging sensor or (ii) an alignment of the imaging sensor relative to the imaging device. In some embodiments, the alignment system is configured to calibrate the imaging sensor relative to the imaging device. In some embodiments, the system further comprises one or more sensors configured to provide feedback on (i) an alignment of the imaging device relative to the imaging sensor or (ii) an alignment of the imaging sensor relative to the imaging device. In some embodiments, the system further comprises an alignment system configured to automatically adjust (i) the alignment of the imaging device relative to the imaging sensor or (ii) the alignment of the imaging sensor relative to the imaging device, based on one or more measurements obtained using the one or more sensors.

In some embodiments, the optical adapter further comprises a connection interface integrated with a housing of the optical adapter, wherein the connection interface is configured to releasably couple the optical adapter to an eyepiece of (i) the imaging device and/or (ii) focusing optics integrated with the imaging device. In some embodiments, the imaging device comprises the human eye. In some embodiments, the at least one prism comprises a Penchant roof prism. In some embodiments, the optics assembly comprises at least one achromatic doublet combined with a singlet to reduce spherical and chromatic aberrations induced or created by the at least one prism. In some embodiments, the imaging sensor is configured for laser speckle imaging of the surgical scene. In some embodiments, the first optic is configured to receive parallel beams of the visible light from the scope and transmit the parallel beams of the visible light to the second optic.

In another aspect, the present disclosure provides an adaptor comprising the system of any aspect or embodiment of a system disclosed herein. In some embodiments, the adaptor is releasably couplable to an imaging device and releasably couplable to a scope.

In another aspect, the present disclosure provides a method comprising providing the system of any aspect or embodiment disclosed herein.

In another aspect, the present disclosure provides a method comprising providing the adaptor of any aspect or embodiment disclosed herein; coupling the adaptor to an imaging device; and coupling the adaptor to a scope. In some embodiments, the method further comprises aligning the adaptor relative to the scope. In some embodiments, the method further comprises aligning the imaging device relative to the adaptor.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A and FIG. 1B schematically illustrate various implementations of coupling an imaging device of the present disclosure to a scope of the present disclosure.

FIG. 2A and FIG. 2B illustrate an example of an optical adapter coupled to various imaging devices of the present disclosure.

FIG. 3 illustrates an example of an optical layout of optical adapter comprising an imaging sensor, in accordance with some embodiments.

FIG. 4 schematically illustrates an optic layout 400 of a prism-based design comprising a pechan prism, in accordance with some embodiments.

FIG. 5 schematically illustrates an optic layout 500 of a prism-based design comprising a porro prism, in accordance with some embodiments.

FIG. 6A and FIG. 6B illustrate an example beam path of an image through a pechan prism pair, in accordance with some embodiments.

FIG. 7A illustrates a 3D ray path diagram of a double porro prism system, in accordance with some embodiments.

FIG. 7B illustrates a 3D ray path diagram of an Abbe-Porro prism system, in accordance with some embodiments.

FIG. 8A schematically illustrates an exploded view of a system comprising a prism, in accordance with some embodiments.

FIG. 8B schematically illustrates an isomorphic view of a system comprising a prism, in accordance with some embodiments.

FIG. 9 schematically illustrates an optic layout comprising a double relay system.

FIG. 10A schematically illustrates a section view of a device comprising a lens-based optic layout, in accordance with some embodiments.

FIG. 10B schematically illustrates an external isomorphic view of a device comprising a lens-based optic layout, in accordance with some embodiments.

FIG. 11 schematically illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out.

The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

### Optical Adapter

In an aspect, the present disclosure provides optical adapters that are capable of imaging in multiple modalities, and universally compatible with any type of imaging device, regardless of hardware configuration and/or form factor.

The optical adapter may be used to image a scene (e.g., a surgical scene). The optical adapter may enable multi-wavelength and/or hyperspectral imaging of the scene. The optical adapter may be used to image a surgical scene using visible light and/or non-visible light. In some embodiments, the optics assembly may be configured to actively or passively separate light based on wavelength. In some embodiments, the optical adapter may be configured for multiple uses. In some embodiments, the optical adapter may be configured for single use.

The optical adapter may be attachable to a scope and/or an imaging device (e.g., a third-party camera). In some cases, the optical adapter may be attachable to focusing optics integrated with the imaging device. In some cases, the optical adapter may be configured to pass white light through an optics assembly to the imaging device coupled to the optical adapter.

In some embodiments, the optical adapter may comprise a sealed housing comprising one or more windows for receiving an input optical beam comprising visible light and/or the non-visible light. The input optical beam may be transmitted through a scope that is attached to the optical adapter. The input optical beam may be provided to various optics or optics assemblies in the optical adapter. In some embodiments, the optical adapter may comprise a channel for confining or controlling a divergence of the input optical beam. In some cases, the channel may comprise a high refractive index material. The channel may be in optical communication with an optics assembly of the optical adapter, an optical element of the optical adapter, and/or an imaging device or an imaging sensor that is integrated with and/or attached to the optical adapter or portion thereof.

Imaging device—In some embodiments, the optical adapter may be attachable to an imaging device. The imaging device may comprise an external or third-party camera. In some cases, the imaging device may comprise an imaging sensor for visible light imaging of the surgical scene. In some cases, the imaging device may comprise a human eye.

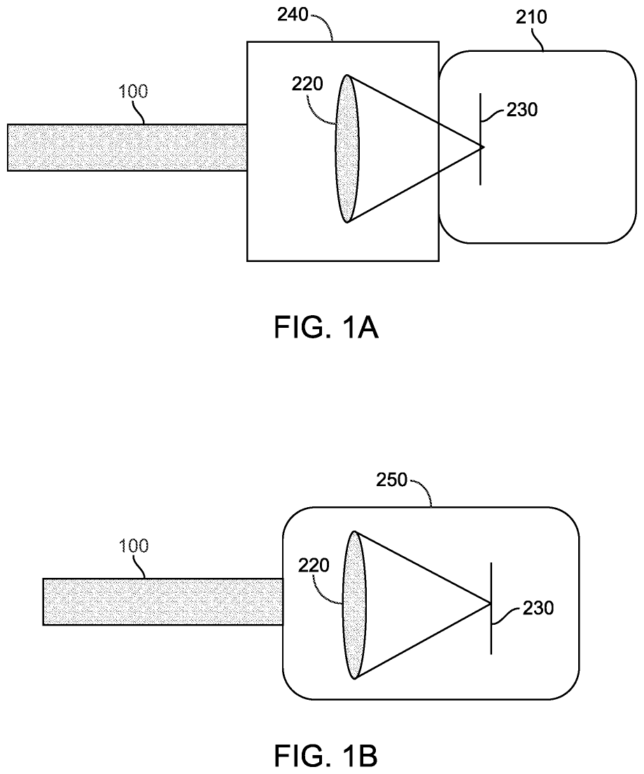

FIG. 1A and FIG. 1B schematically illustrate various implementations of coupling an imaging device of the present disclosure to a scope of the present disclosure. An imaging device may be coupled to a scope in various ways. For example, a scope may be connected to a C-Mount camera with a coupler or two a camera with an integrated couple. Each type of imaging device may comprise a detector 230 and a focusing optic 220. The focusing optic may facilitate focusing of the image onto the detector. The focusing optic may account for a depth of focus for a particular imaging device. In some cases, the detector 230 comprises a CCD, a CMOT, or another device that converts optical signal to image data.

FIG. 1A schematically illustrates a scope 100 coupled to an imaging device comprising a C-Mount camera 210 and a coupler 240 for a C-mount imaging device. A C-mount may be a type of lens mount found on cameras. A C-Mount scope in the laparoscopy setting may be called a direct view scope. A C-Mount scope may create a contained environment from the scope directly to the camera head using an O-ring. The O-ring may prevent water from entering view during a procedure. When using a coupler, water may come between the scope and the coupler. This may create a fog or distortions in the image, making it more difficult for a surgeon. In some cases, a surgeon may have to wipe down the eyepiece of the scope and the camera head throughout the procedure. This may extend surgical timelines. Despite this possibility, C-mount scopes are used during laparoscopy. These scopes may be more compatible with multiple manufacturer camera heads across different specialties.

FIG. 1B schematically illustrates a scope 100 coupled to an imaging device with an integrated coupler 250. Integrated couplers remove the second component. They may be used with eyepiece scopes. The single connection point between the scope and the integrated coupler removes a condensation point during procedures. The integrated coupler may allow for sterilization of one product (coupler and camera head at the same time). C-Mount scopes cannot generally be used with integrated camera heads.

Scope—In some embodiments, the optical adapter may be attachable to a scope 100 and an imaging device. The scope and the imaging device may be provided separately from the optical adapter. The optical adapter may be compatible with any type of scope or imaging device, regardless of hardware configuration and/or form factor. A scope of the present disclosure may be a scope system used in minimally invasive surgery. A scope of the present disclosure may be a laparoscope, an arthroscope, an endoscope, a proctoscope, a rectoscope, etc. In some cases, the scope may not be used in a medical setting. For example, the scope may be a borescope.

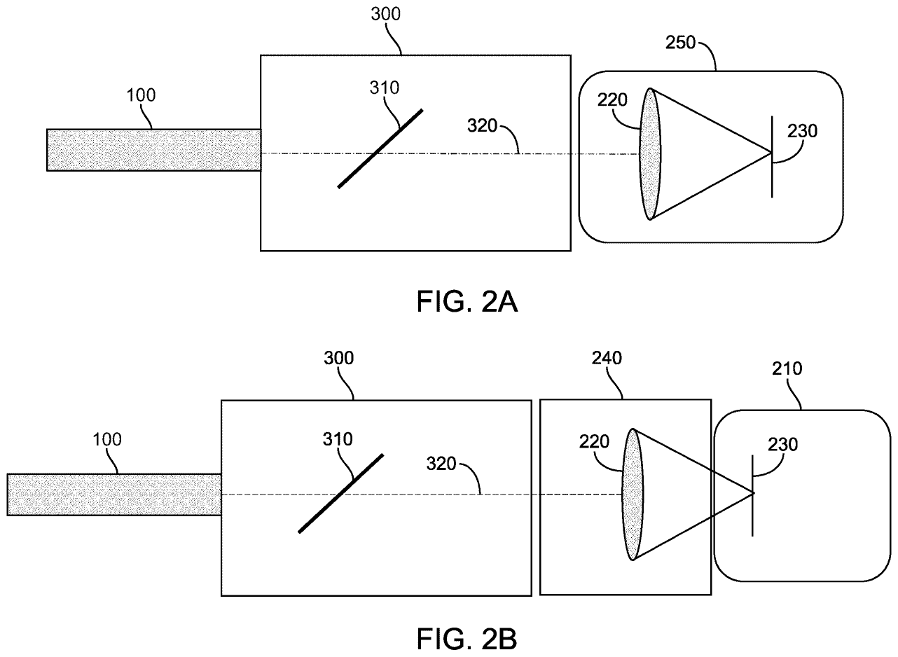

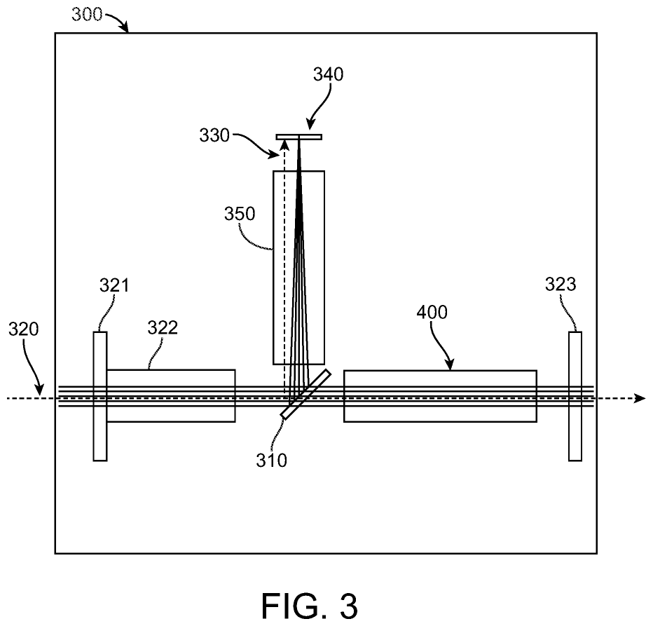

FIG. 2A and FIG. 2B illustrate an example of an optical adapter 300 coupled to various imaging devices of the present disclosure. FIG. 2A schematically illustrates an optical adaptor 300 connected to an imaging device with an integrated coupler 250. FIG. 2B schematically illustrates an optical adaptor 300 connected to an imaging device comprising a C-Mount camera 210 and a coupler 240 for a C-mount imaging device. The optical adapter 300 may be configured to couple to or interface with a surgical scope 100. In some cases, a surgical scope 100 may be releasably coupled to the optical adapter 300. The optical adapter 300 may comprise a connection interface for coupling the scope 100 to the optical adapter 300. In some cases, the connection interface may be configured to interface with a plurality of different types of scopes having different sizes, shapes, form factors, or hardware configurations.

In some embodiments, the optical adapter 300 may be configured to couple to an imaging device. The imaging device may comprise a third-party camera that is provided separately from the optical adapter 300. In some cases, the optical adapter 300 may comprise a connection interface for coupling the imaging device to the optical adapter 300. The connection interface may comprise, for example, an eyepiece connection interface. The eyepiece connection interface may allow for coupling of the optical adapter 300 directly to an eyepiece of the third-party camera.

In an example of the imaging device of FIG. 2B, the eyepiece connection interface may allow for coupling of the optical adapter 300 directly to a coupler 240. A coupler may comprise a focusing mechanism of the third-party camera. The focusing mechanism may comprise an adjustor which translates the focusing optic along the optical axis 320 to adjust a focal depth of the image on the detector 230.

In an example of the imaging device of FIG. 2A, the eyepiece connection interface may allow for coupling of the optical adapter 300 directly to the scope 100. An integrated coupler may comprise a focusing mechanism of the third-party camera. The focusing mechanism may comprise an adjustor which translates the focusing optic along the optical axis 320 to adjust a focal depth of the image on the detector 230.

The optical adapter 300 may be configured to interface with a scope 100 and an imaging device. As described elsewhere herein, in some cases the optical adapter 300 may be configured to interface with focusing optics 220 associated with the detector 230. The focusing optics 220 may be integrated with the imaging device such that the focusing optics 200 for the camera need not be built in or integrated with the optical adapter 300, thereby simplifying the design of the optical adapter 300 and enhancing the compatibility of the optical adapter 300 with camera systems or other imaging devices having their own built in focusing optics/focusing mechanisms.

In some embodiments, the optical adapter 300 may comprise one or more optics that collectively function as a passive adapter to transmit image signals from the scope 100 to the imaging device and/or an imaging sensor integrated with the optical adapter 300, without distorting the image received at the imaging device. In some embodiments, the image signal received at the detector 230 may substantially correspond to the image signal received by or from the scope 100.

In some cases, the visible light exiting the optics assembly toward the imaging device comprises one or more of: about the same image size, about the same image collimation, or about the same image orientation as the visible light entering the optics assembly from the scope. About the same may mean a range of up to 20%, up to 10%, up to 5%, or up to 1% of a given value. For example, about the same image size, about the same image collimation, or about the same image orientation may mean less than 20% difference between the visible light entering and exiting the optics assembly from the scope

In some cases, the visible light exiting the optics assembly toward the imaging device comprises one or more of: substantially the same image size, substantially the same image size image collimation, and substantially the same image size image orientation as the visible light entering the optics assembly from the scope. Substantially the same collimation may be sufficiently the same such that the image may be focused on the detector without changes in focus outside the tolerances of a standard focus adjustment. Substantially the same image size may be sufficiently the same such that a user would not choose to zoom in or out on the software beyond standard software tolerances. Substantially the same image orientation may be sufficiently the same such that the image does not need to be flipped, mirrored, or inverted by the software of the imaging device.

The image signal may comprise one or more signals (e.g., optical signals) that correspond to an image or a view of the surgical scene from the perspective of the scope. The one or more signals may comprise one or more beams or photons of light. The image signal may comprise one or more signals that can be used to generate an image of the surgical scene from the perspective of the scope or provide a representation of a view of the surgical scene from the perspective of the scope. In some cases, the image signal may be produced at the output of the scope as the scope is being used to image the surgical scene or any objects or features that are detectable or present therein.

In a clinical setting, it may be important that an orientation of the image is substantially the same at the entrance and the exit because it may pose a danger to patients if the image on the imaging device is not the same orientation as what a physician expects. Preserving image size and image collimation may facilitate coupling multiple types of imaging devices with a scope of a present disclosure. For example, if the light exiting the adaptor has about the same image size and image collimation, imaging devices (which may be designed for the image characteristics exiting the scope) may be generally more compatible. However, since adding the adaptor adds beam path, it may be difficult to preserve image characteristics while maintaining a device with a small form factor and relative optical simplicity.

Imaging sensor—In some embodiments, the optical adapter may comprise an imaging sensor. A second imaging sensor may allow for additional functionality. For example, an imaging sensor may allow for a second imaging modality. An adaptor of the present disclosure may allow for a second imaging modality to be integrated with a scope system including an imaging device and a scope which may already be use in a clinical setting. In some cases, an imaging sensor may be configured for laser speckle imaging of the surgical scene. The imaging sensor may comprise, for example, a sensor configured to detect non-visible light. Such non-visible light may comprise, for example, infrared light. The infrared light may include near IR light, mid IR light, and/or far IR light. The imaging sensors may be built in or integrated with the optical adapter.

FIG. 3 illustrates an example of an optical layout of optical adapter 300 comprising an imaging sensor 340. The imaging sensor 340 may be integrated with or coupled to the optical adapter 300 or a housing of the optical adapter. In some embodiments, the optical adapter 300 may comprise an optical element 303. The optical element 310 may be configured to receive light 320 from a scope 100 that is coupled to the optical adapter 300 and split that light into two beam paths. The light received from the scope may comprise visible light and non-visible light. In some cases, the optical element 310 may be configured to direct the visible light to the imaging device and the non-visible light 330 to the imaging sensor 340. In some cases, the optical element 310 may comprise a beam splitter. In some cases, the optical element 310 may comprise a dichroic mirror or lens. In any of the embodiments described herein, the optical element 310 may be configured to split the visible light and the non-visible light based on wavelength. In some cases, the optical element 310 may be configured to direct light having a wavelength that is greater than a threshold wavelength towards the imaging sensor. In some cases, the optical element 310 may be configured to direct light having a wavelength that is less than a threshold wavelength towards the imaging device or camera. The threshold wavelength may range from about 700 nanometers to about 800 nanometers (nm). In other embodiments, the threshold wavelength may range from 500 nm to 2000 nm. In further embodiments, the optical element 310 may be configured to direct light having a wavelength range to form a bandpass or a notch filter, towards the imaging sensor, with the complementary light transmitted in the white light direction.

In some cases, optical element 310 comprises a dichroic beam splitter with a threshold wavelength of 750 nm. The reflected wavelength range may be about 750 nm to 950 nm. The transmitted wavelength range may be about 400 nm to 750 nm.

Optical element—As described elsewhere herein, in some embodiments, the optics assembly may comprise an optical element configured to direct the visible light to the imaging device and the non-visible light to the imaging sensor. In some cases, the optical element may comprise a beam splitter. In some cases, the optical element may comprise a dichroic mirror or lens.

In some cases, the optical element may be positioned upstream of the first optic. In some cases, the optical element may be positioned between the first optic and the third optic. In some cases, the optical element may be positioned downstream of the third optic.

The optical layout may comprise an entrance window 312 and an exit window 323. The entrance window and the exit window may facilitate sealing the adaptor. The input window may be coupled to a confinement tunnel 322. The confinement tunnel may support one or more optical elements. The confinement tunnel may help limit external light contamination. The confinement tunnel may help limit light leakage into the environment. In some cases, the windows may comprise glass windows, BK-7 windows, quartz windows, sapphire windows, etc. The thickness of the windows may be less than 10 millimeters (mm) and greater than 0.5 mm. The thickness of the windows may be about 2 mm.

The optical layout may comprise an optics assembly 400 for directing light to the imaging device and an optics assembly 350 for directing light to the imaging sensor 340. In some embodiments, the optical adapter may comprise an optics assembly for directing (i) the visible light to the imaging device and (ii) the non-visible light to the imaging sensor.

### Optics Assembly

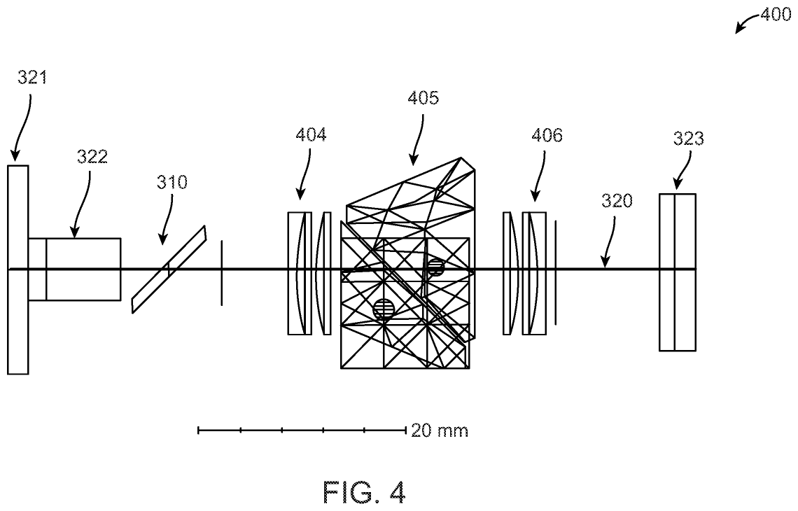

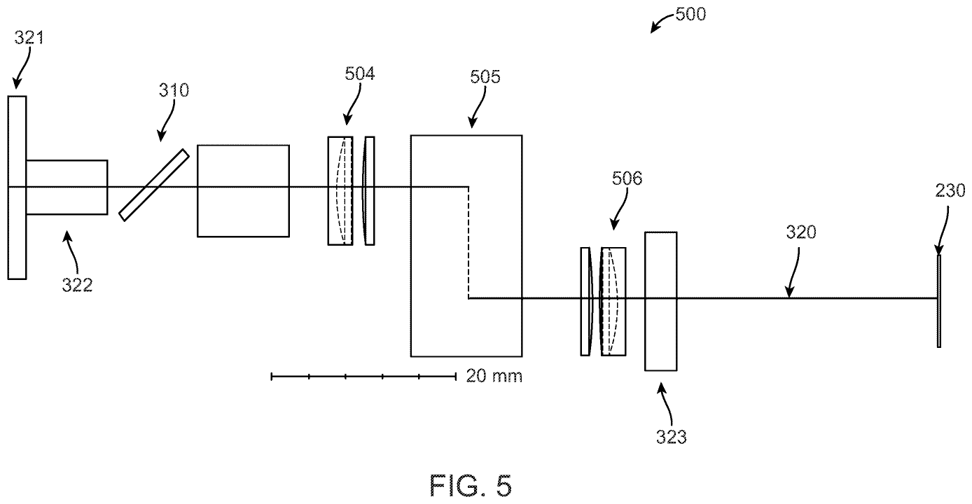

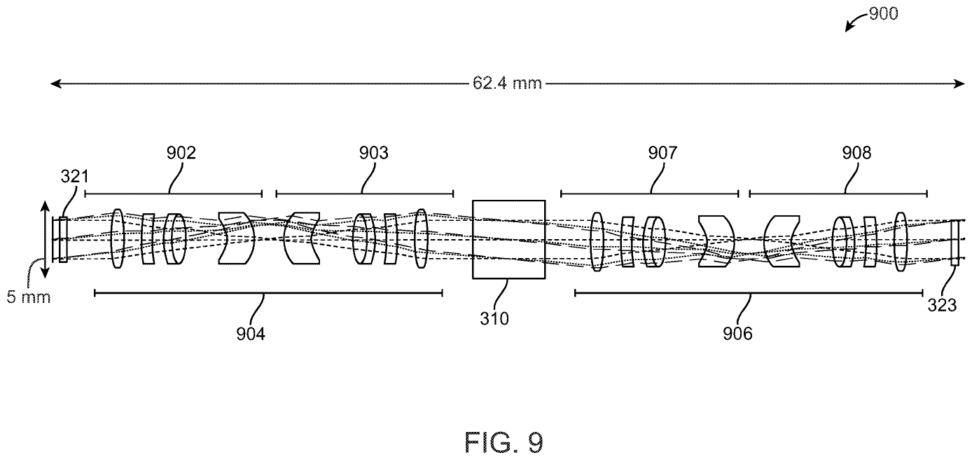

FIG. 4, FIG. 5, and FIG. 9 schematically illustrate various optic layouts of an optics assembly for directing light to an imaging device of the present disclosure. Each of FIG. 4, FIG. 5, and FIG. 9 show optical layouts which may preserve the image size, image collimation, and image orientation of the light directed from a scope to an imaging device.

FIG. 4 and FIG. 5 are prism-based designs. FIG. 9 comprises a design without a prism. The example of FIG. 9 may act as a double relay. The design without a prism may comprise more optical elements. A double relay lens system may comprise lens element focal lengths which are comparatively short, so surfaces of lens elements may have to be aspheric in order to compensate for shorter focal lengths. Double relay system may comprise higher cost and tighter tolerances for the optical components than the prism-based design. However, the double relay design may be useful in situations where it is advantageous to invert the image on the imaging sensor. Both designs have the advantage of a relatively small lengths scale.

Turning to the prism-based designs, in some embodiments, the optics assembly may comprise a first optic, a second optic, and third optic. FIG. 4 schematically illustrates an optic layout 400 of a prism-based design comprising a pechan prism. FIG. 5 schematically illustrates an optic layout 500 of a prism-based design comprising a porro prism. In some embodiments, the second optic may be positioned between the first optic and the third optic. In some embodiments, the first optic may be configured to direct at least a portion of the visible light to the second optic. In some embodiments, the second optic may be configured to manipulate the visible light to adjust a property or a characteristic of an image associated with or derivable from the visible light. In some embodiments, the third optic may be configured to receive the visible light from the second optic and provide parallel beams of the visible light directly to the imaging device. In some embodiments, the parallel beams of the visible light may substantially replicate an image signal that is received by or from the scope.

First Optic: In some embodiments, the first optic may be configured to receive non-parallel or parallel beams of the visible light from the scope. The first optic may be configured to transmit the non-parallel or parallel beams of the visible light to the second optic. In some cases, the first optic may be a first lens assembly 404 of 504. As shown, the first lens assembly may adjust a focal characteristic, a magnification, or both of the light coming from the beam splitter to the imaging device. A focal characteristic may comprise an image collimation. For example, light may be collimated (e.g., have substantially parallel beams), be converging, or be diverging. Each lens of the lens assembly may change a collimation of the light. A combination of a pair of lenses may be spaced such that the net effect of the lens pair is to maintain image collimation while increasing or decreasing magnification (e.g., image size). The magnification is related to the relative focal lengths of each lens when spaced at an appropriate distance to maintain collimation.

Second Optic: The second optic may be configured to receive the non-parallel or parallel beams of the visible light from the first optic. In some cases, the second optic may be configured to manipulate the visible light to adjust a property or a characteristic of an image associated with or derivable from the visible light. The second optic may be configured to transmit the manipulated beams of the visible light to the third optic. The second optic may comprise one or more prism systems 405 or 505.

Third Optic: In some embodiments, the third optic may be configured to receive the visible light beams from the second optic and provide one or more parallel beams of the visible light directly to the imaging device. The one or more parallel beams of the visible light may be produced from the visible light beams received from the second optic. The visible light beams received from the second optic may correspond to or may comprise one or more beams of visible light manipulated by the second optic. In some cases, the third optic may be a second lens assembly 406 of 506. The second lens assembly may adjust a focal characteristic, a magnification, or both of the light coming from the beam splitter to the imaging device.

In some cases, the parallel beams of the visible light may form a nominally collimated beam. In some cases, the parallel beams of the visible light may form a nominally collimated beam with a small amount of divergence or convergence that is proportional to the nominally collimated beam divergence or convergence produced by the scope 100 or any other device coupled to optical adapter 300. In some cases, the nominally collimated beam may be usable to generate an RGB or visible light image of the surgical scene. The RGB or visible light image of the surgical scene may not be inverted, rotated, or visually distorted relative to the surgical scene as viewed from or through the scope (which may be positioned upstream of the first optic). In some cases, the parallel beams of the visible light may be focused on a plurality of different regions of a light sensing unit of the imaging device.

In some embodiments, the parallel beams of the visible light may be usable to generate an output image having a same property or characteristic as a reference image associated with the image signal received by or from the scope. In some cases, the property or characteristic may comprise an image orientation, an image quality, or an image fidelity. In some cases, the image signal at the scope may be replicated without post-processing of the output image generated from the parallel beams of the visible light.

FIG. 5 schematically illustrates an optic layout 500 of a prism-based design comprising a porro prism. The optics assembly 500 may comprise an input window 321 and a tunnel 322 in optical communication with the input window. An input beam from a scope or a surgical scene may be received by the optical adapter through the input window 321 and transmitted to a beam splitter 310 via the tunnel 322, which may be configured to confine or control the divergence of the input beam.

In some cases, the beam splitter 310 may be configured to direct infrared light to an imaging sensor of the optical adapter (e.g., for laser speckle imaging). In some cases, the beam splitter 310 may be configured to direct white light or visible light to the first lens assembly 504.

In some cases, the first lens assembly 504 may be configured to direct the white light to a prism 505. The prism 505 may comprise a Porro prism. The Porro prism may be configured to fold an optical path of the visible light and manipulate (e.g., invert, rotate, or mirror) an image associated with the visible light in order to substantially replicate an image signal associated with the input light beam received by or from a scope. In some cases, the Porro prism may be configured to direct the visible light beams to a second lens assembly 506 and/or an output window 323 that is aligned with or in optical communication with an imaging device (e.g., a third-party camera). In some embodiments, the first lens assembly 504 and the second lens assembly 506 may comprise symmetrical lens sets.

Turning to lens-based designs, FIG. 9 schematically illustrates an optic layout 900 comprising a double relay system. As shown, optic layout 900 may comprise two afocal relays 904 and 906, each symmetric about their internal image. Each afocal relay may comprise a lens assembly of the present disclosure. The first afocal relay 904 inverts the image. The second afocal relay 906 reverses an image flip from the first afocal relay. A symmetrical relay design may reduce aberrations (e.g., coma, distortion, lateral contour, etc.). The double relay system may comprise four repeated lens cells 902, 903, 907, and 908 which collectively comprise the optic layout 900.

Optical layout 900 may also comprise an optical element 310 configured to direct the visible light to the imaging device and the non-visible light to the imaging sensor. In some cases, the optical element may comprise a beam splitter. In some cases, the optical element may comprise a dichroic mirror or lens. Unlike the prism-based optical layouts, optical element 310 is between the first afocal relay and the second afocal relay rather than preceding the first optic, second optic, and third optic. Because the image after the first afocal relay is inverted, the image directed to the imaging sensor may also be inverted. While in the prism-based designs, the image directed to the imaging sensor may not be inverted. In optical layout 400, 500, and 900, the image directed to the imaging device may not be inverted.

Lens Assembly—In some embodiments, the optics assembly may comprise one or more lenses or lens assemblies. For example, the optics assembly may comprise a first lens assembly and a second lens assembly. In some embodiments, the at least one prism may be disposed between the first lens or lens assembly and the second lens or lens assembly. In some embodiments, the first lens or lens assembly and the second lens or lens assembly may be provided in a symmetrical configuration relative to the at least one prism. In some cases, at least one of the first lens or lens assembly and the second lens or lens assembly may comprise a symmetrical lens.

In some embodiments, the one or more lenses may comprise at least one achromatic lens to reduce spherical and/or chromatic aberrations induced or created by the at least one prism and any other optical component that is a part of, integrated with, or connected to the optical adapter 300. In some cases, the at least one achromatic lens may comprise an achromatic singlet lens or an achromatic doublet lens. In some embodiments, the optics assembly may comprise at least one achromatic doublet combined with a singlet to reduce spherical and chromatic aberrations induced or created by the at least one prism. In some cases, the optics assembly may be configured to remove or reduce aberrations in one or more output images generated using the parallel beams of the visible light. In some cases, the optics assembly may be optically symmetric relative to the at least one prism to remove or reduce odd order aberrations.

In some embodiments, the one or more lenses may comprise a telecentric lens. In some embodiments, the optics assembly may be configured to provide a telecentric pupil space.

Prism—In some embodiments, the optics assembly may comprise at least one prism. As described elsewhere herein, the optics assembly may comprise at least one prism. In some cases, the at least one prism may comprise a roof prism. In some cases, the roof prism may comprise a Penchant roof prism. In some embodiments, the at least one prism may comprise a Porro prism. In any of the embodiments described herein, the at least one prism may be configured to fold an optical path of the visible light. In some embodiments, the at least one prism may be displaced or offset to compensate for a shift in an optical path or axis of the visible light that is induced or caused by one or more components or sub-components of the optics assembly.

In some cases, the second optic 405 or 505 is a prism system. In some cases, a prism system may be a single prism. In some cases, a prism system may be a prism pair. In some cases, a prisms system may comprise a plurality of prisms. For example, a prism system may comprise a pechan prism pair, a porro prism pair, an Uppendahl prism system, an Abbe-Porro, or an Abbe-Koenig prism system. In some cases, the prism is an image erector. In some cases, the prism inverts the image along an optical path from one side of the prism system to the other.

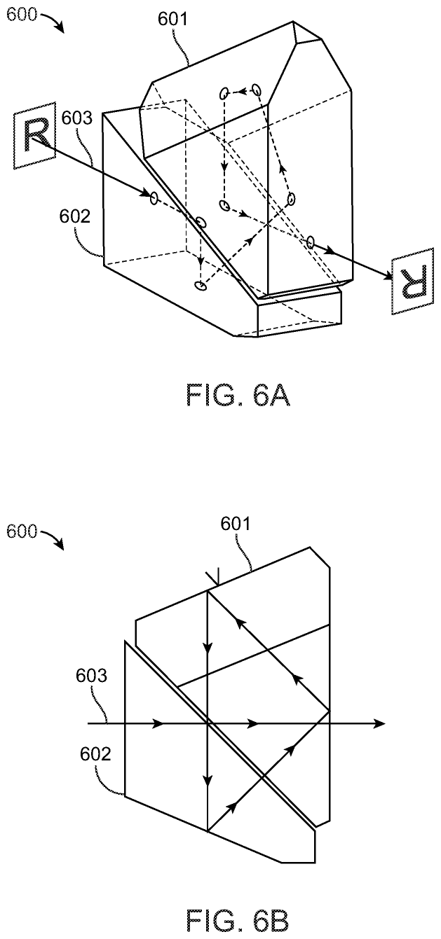

FIG. 6A and FIG. 6B illustrate an example beam path of an image through a pechan prism pair 600. FIG. 6A illustrates a 3D ray path diagram. FIG. 6B illustrates a 2D ray path diagram. A pechan prism system 600 may be comprised of a Schmidt prism 601 and a half-penta prism 602. A pechan prism system may comprise a beam path 603 with six reflections and a small air gap to allow for TIR inside the prism system. The even number of reflections may enable the image to stay right-handed. No or a relatively small displacement is produced along the object's axis assuming proper alignment. The input beam and the output beam may be substantially co-axial. As shown, the image is inverted from one side of the prism to another. The net effect may be to flip the image in both the horizontal and vertical axes.

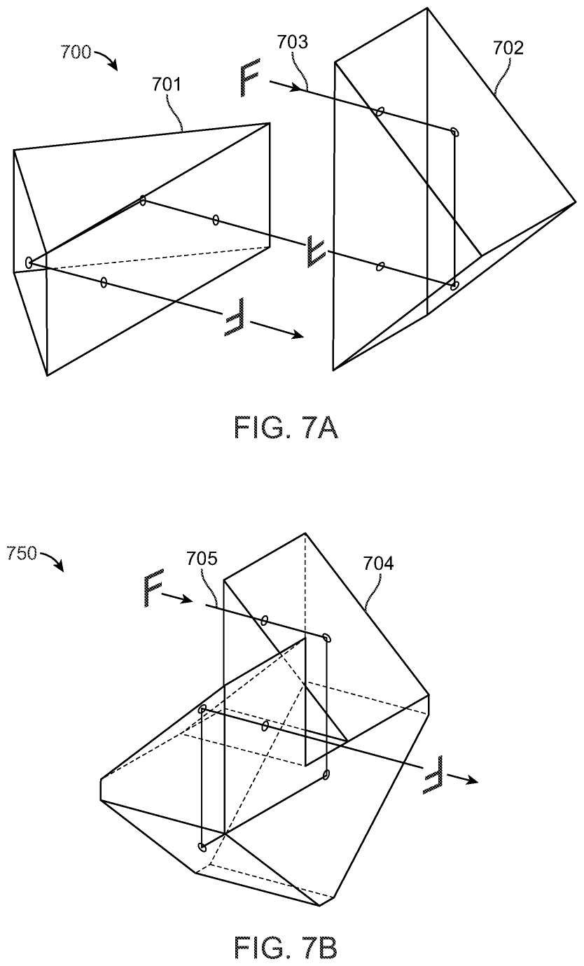

FIG. 7A illustrates a 3D ray path diagram of a double porro prism system 700 comprising an air gap. The double porro prism system may comprise a first porro prism 701 and a second porro prism 702. As shown, the double porro prism system may comprise a light path 703. A porro prism pair may comprise a beam path with a total of four reflections. The even number of reflections may enable the image to stay right-handed. As shown, the image is inverted from one side of the prism to another. The net effect may be to flip the image in both the horizontal and vertical axes. As shown, there is a translation of the image in both the horizontal and vertical axes. The entrance and exit beam axes may be substantially parallel.

FIG. 7B illustrates a 3D ray path diagram of an Abbe-Porro prism system 750 comprising no air gap. An Abbe-Porro prism system may a comprise a single optical element 704. As shown, the Abbe-Porro prism may comprise a light path 705. Light enters one flat face, is internally reflected four times from the sloping faces of the prism, and exits the second flat face offset from, but in the same direction as the entrance beam. The Porro-Abbe system may reduce the lateral beam axis offset by 23% compared to a double Porro prism system. The entrance and exit beam axes may be substantially parallel. The even number of reflections may enable the image to stay right-handed. As shown, the image is inverted from one side of the prism to another. The net effect may be to flip the image in both the horizontal and vertical axes.

As described elsewhere herein, the optics assembly may comprise a plurality of optics. The plurality of optics may comprise the first optic, the second optic, and the third optic as described above. In some cases, the first optic may comprise a first lens or a first lens assembly comprising the first lens. In some cases, the second optic may comprise the at least one prism. In some cases, the third optic may comprise a second lens or a second lens assembly comprising the second lens.

In some cases, the first lens or lens assembly may be configured to produce the image associated with or derivable from the visible light inside or within the at least one prism. In some cases, the at least one prism may be configured to manipulate the image associated with or derivable from the visible light received from the first lens or lens assembly in order to replicate the image signal received by or from the scope. In some cases, the second lens or lens assembly may be configured to receive the manipulated visible light beams from the at least one prism and direct the parallel beams of the visible light to the imaging device. In some cases, the parallel beams of the visible light may correspond to the image manipulated by the at least one prism.

In some embodiments, the optics assemblies disclosed herein may be configured to eliminate or prevent a formation, projection, or placement of one or more intermediate image planes on a portion, surface, or edge of the at least one prism. In some cases, the optics assemblies may be configured to provide an output signal that substantially maintains a quality of the image signal received by or from the scope.

In some embodiments, the optics assemblies may be configured to diverge light received by the optical adapter into a plurality of paths based on wavelength. In some embodiments, the optics assemblies may be configured to separate wavelengths of light without distorting the image signal received by or from the scope. In some embodiments, the optics assemblies may be configured to flip or rotate an RGB image derivable from the visible light in order to replicate the image signal received by or from the scope.

In some embodiments, the optics assemblies may be configured to provide or maintain a constant optical axis for one or more optical signals received by the optical adapter and/or transmitted to the imaging device. In some cases, the constant optical axis may extend from a first end of the optical adapter to a second end of the optical adapter. In some embodiments, the imaging device may be positioned at the second end of the optical adapter. In some cases, the imaging axis of the imaging device may be aligned with the constant optical axis extending through the optical adapter.

In some embodiments, the optics assembly may be configured to manipulate the image associated with or derivable from the visible light in order to replicate the image signal that is received by or from the scope. In some embodiments, the optics assembly may be configured to manipulate the image associated with or derivable from the visible light by rotating, reorienting, flipping, mirroring, inverting, or resizing the image.

Materials—The optics and optical elements described herein may comprise one or more materials. The one or more materials may be selected to optimize performance and/or to minimize manufacturing costs. In some cases, the one or more materials may comprise a plastic. In some cases, the one or more materials may comprise a polycarbonate. In some cases, the one or more materials may comprise glass.

### Example Configurations

The optic layouts disclosed herein may be integrated into an imaging system as described here. The imaging system may comprise an optical adapter as described elsewhere herein. The imaging system may comprise a housing.

Alignment system—In some embodiments, the system may comprise an alignment system. The alignment system may comprise a mechanical alignment device or a software-based alignment algorithm.

In some cases, the alignment system may be configured to adjust (i) an alignment of the imaging device relative to the imaging sensor or (ii) an alignment of the imaging sensor relative to the imaging device. In some cases, the alignment system may be configured to calibrate the imaging sensor relative to the imaging device.

In some cases, the system may comprise one or more sensors configured to provide feedback on (i) an alignment of the imaging device relative to the imaging sensor or (ii) an alignment of the imaging sensor relative to the imaging device. In some cases, the system may comprise one or more alignment systems configured to automatically adjust (i) the alignment of the imaging device relative to the imaging sensor or (ii) the alignment of the imaging sensor relative to the imaging device, based on one or more measurements obtained using the one or more sensors.

The alignment system may be configured to align the imaging sensor relative to the imaging device (e.g., the third-party camera). The alignment system may be configured to adjust an alignment (e.g., a position or an orientation) of the imaging sensor relative to the imaging device.

In some embodiments, the alignment system may comprise one or more alignment features. The one or more alignment features may comprise pegs, détentes, alignment indicators, etc. The one or more pegs may correspond to one or more slots or recesses on the imaging device. The one or more pegs may be configured to interface with the one or more slots or recesses and mechanically adjust a rotation or an orientation of the imaging device.

In some embodiments, the alignment system may reduce a likelihood that an imaging device can rotate or move relative to the adaptor once the systems are coupled.

In some cases, the alignment system may be configured to perform active alignment. The active alignment may involve the use of software and sensors to provide constant feedback on the positional alignment of two or more imaging devices or sensors and calibrate the imaging devices or sensors accordingly.

Connection Interface—In some embodiments, the optical adapter may comprise a connection interface integrated with a housing of the optical adapter. In some cases, the connection interface may be configured to releasably couple the optical adapter to an eyepiece of (i) the imaging device and/or (ii) focusing optics integrated with the imaging device.

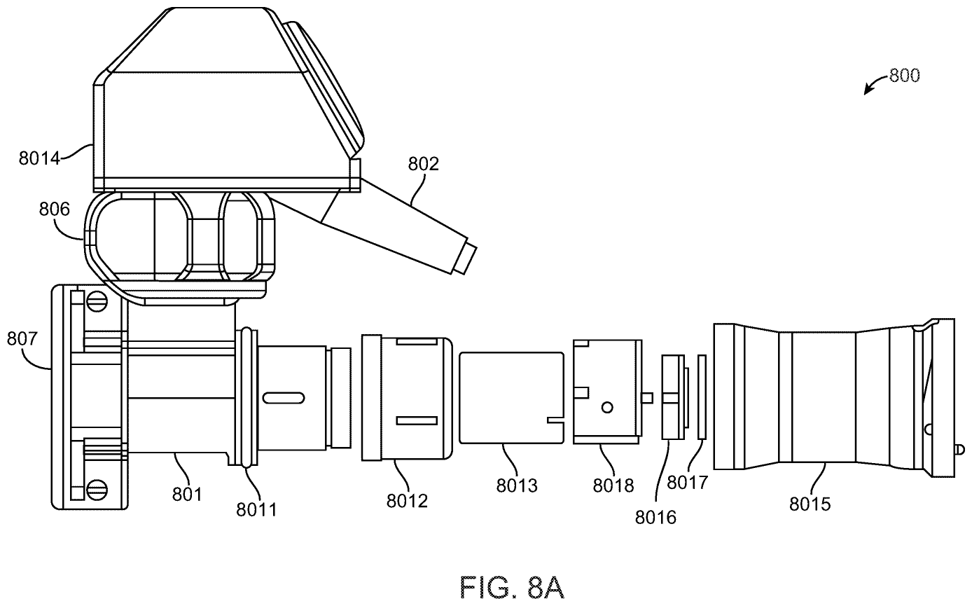

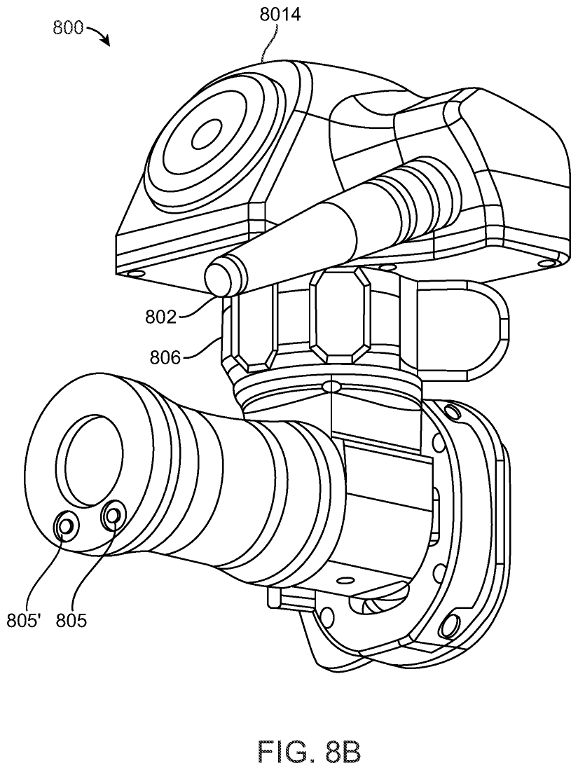

Prism Based Designs—FIG. 8A schematically illustrates an exploded view of a system 800 of the present disclosure. System 800 may be used with a prism-based design such as optic layout 400 or 500 disclosed herein.

The imaging system may comprise an eyepiece sub-assembly. The eye-piece sub-assembly may comprise a housing 8015 configured to contain one or more optical elements. The one or more optical elements may comprise a proximal lens assembly 8016, a prism mount assembly 8018, and a distal lens assembly 8013. The eye-piece subassembly may comprise one or more sealing elements 8011 and 8017 configured to aid in maintaining a seal against liquid ingress into the imaging device. The eye-piece sub-assembly may comprise an eye piece collar 8012. The eye-piece collar may facilitate use of the eye-piece sub-assembly as a viewer in connection with a scope 100 disclosed elsewhere herein. In some cases, imaging module 801 may be releasably couplable to the eye-piece sub-assembly. For example, in a surgical setting, the eye-piece sub-assembly may allow a practitioner to look down the scope into the surgical scene.

The imaging system may comprise an optical adapter as described elsewhere herein. The imaging module 801 and the eye-piece sub-assembly may individually or collectively comprise an adaptor of the present disclosure. For example, an imaging device may be couplable to interface 807 of system 800. Interface 807 may comprise an example of a connection interface disclosed herein. In some cases, the optical adapter may comprise an input window 321 for receiving an input optical beam from a scope that is imaging a surgical scene. In some cases, the optical adapter may comprise a passageway, channel, or conduit 322 for directing the input optical beam towards one or more optics, lenses, lens assemblies, or optical elements. In some cases, the passageway, channel, or conduit may comprise a confinement tunnel comprising a high refractive index material. The confinement tunnel may be configured to confine or control the divergence of the input optical beam.

In some embodiments, the optical adapter may comprise a beam splitter 310 that is in optical communication with the confinement tunnel. The beam splitter may be configured to send IR light towards an imaging sensor of the optical adapter. The beam splitter may be configured to transmit RGB light along a beam path that coincides with an imaging device (e.g., a third-party camera) that is coupled to the optical adapter. In some cases, the beam splitter may be configured to direct the RGB light towards a first lens or lens assembly. In some cases, the beam splitter may be within imaging module 801.

In some embodiments, the optical adapter may comprise a first lens or lens assembly 8016, a prism 8018, and a second lens or lens assembly 8013. The first lens or lens assembly may be configured to produce an intermediate image of the surgical scene inside the prism. The prism may be configured to invert the intermediate image produced by the first lens or lens assembly. The second lens or lens assembly may be configured to produce an output image based on the intermediate image. The output image may be collimated. In some cases, the second lens or lens assembly may be configured to produce parallel beams of light corresponding to the output image. The first lens assembly 8018 may comprise an example or embodiment of first lens assembly 404 or 504 as disclosed herein. The second lens assembly 8013 may comprise an example or embodiment of second lens assembly 406 or 506 as disclosed herein. The prism 808 may comprise an example or embodiment of prism assembly 405 or 505 as disclosed herein.

In some cases, the optical adapter may comprise an output window 323. The output window may be used to direct the parallel beams of light from the second lens or lens assembly to an imaging device (e.g., a third-party camera). The parallel beams of light may correspond to the output image. The output image may be derived from or generated using the parallel beams of light. In some cases, the optical adapter may comprise an optical filter that is positioned adjacent to or in front of the output window.

In some embodiments, the optics assembly may comprise a beam splitting cube. A beam splitting cube may be an example of an optical element 310. The beam splitting cube may be configured to direct non-visible light to an imaging sensor and visible light to an imaging device, as described elsewhere herein. In some cases, the beam splitting cube may comprise a plurality of prisms arranged adjacent or proximal to each other. In some embodiments, the beam cube may be configured to maintain a constant optical axis to aid in optics positioning and/or camera alignment or focusing.

FIG. 8B schematically illustrates an external isomorphic view of a system 800 of the present disclosure. As shown, system 800 may comprise one or more alignment features 805. The one or more alignment features may comprise pegs, détentes, alignment indicators, etc. The one or more pegs may correspond to one or more slots or recesses on the imaging device. The one or more pegs may be configured to interface with the one or more slots or recesses and mechanically adjust a rotation or an orientation of the imaging device. In some embodiments, the alignment system may reduce a likelihood that an imaging device can rotate or move relative to the adaptor once the systems are coupled.

In some cases, system 800 may comprise an alignment system. The alignment system may comprise a mechanical alignment device 806. The mechanical alignment device 806 may move a lens assembly within an optics assembly 350 for directing light to the imaging sensor 340. With the turn of the knob, the lens assembly may translate toward or away from the imaging sensor to adjust a focus of the image on the imaging sensor. System 800 may comprise an imaging sensor assembly 8014. The image sensor assembly 8014 may comprise and imaging sensor disclosed herein. System 800 may comprise a camera cable assembly 802. The camera cable assembly may facilitate coupling of the imaging sensor to a computer system of the present disclosure.

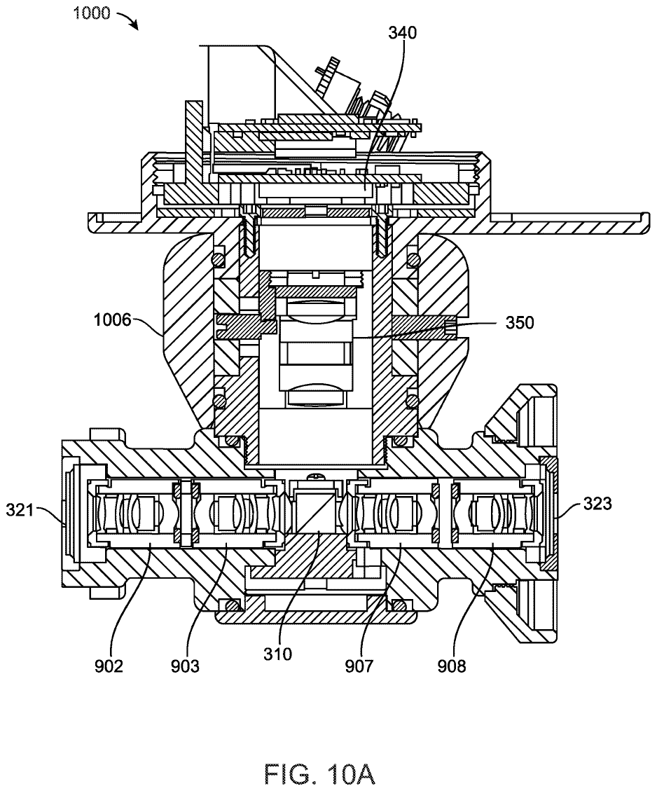

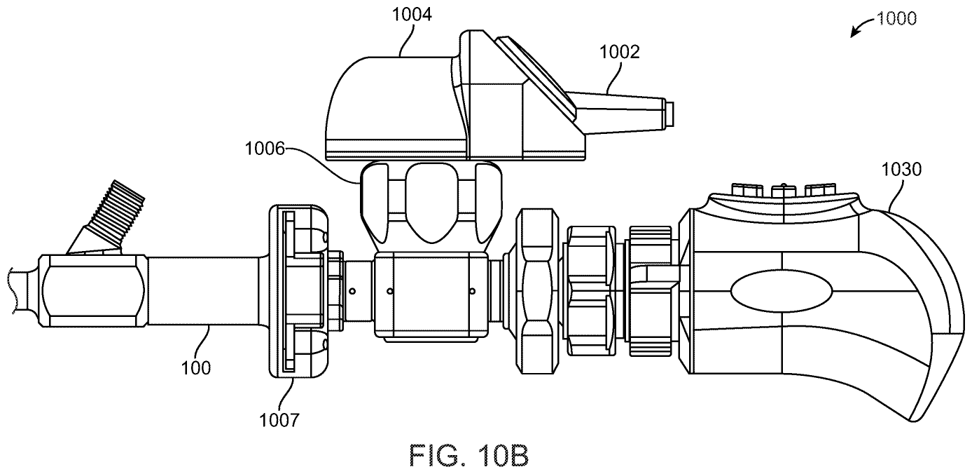

FIG. 10A schematically illustrates a section view of a device 1000 comprising a lens-based optic layout, in accordance with some embodiments. As shown, device 1000 may comprise two afocal relays 904 and 906, each symmetric about their internal image. Each afocal relay may comprise a lens assembly of the present disclosure. Device 1000 may comprise four repeated lens cells 902, 903, 907, and 908 which collectively comprise the optic layout 900.

Device 1000 may also comprise an optical element 310 configured to direct the visible light to the imaging device and the non-visible light to the imaging sensor. In some cases, the optical element may comprise a beam splitter. In some cases, the optical element may comprise a dichroic mirror or lens. Device 1000 may comprise an optics assembly 350 for directing light to the imaging sensor 340. Device 1000 may comprise an entrance window 312 and an exit window 323. The entrance window and the exit window may facilitate sealing the adaptor.

FIG. 10B schematically illustrates an external isomorphic view of a device 1000 comprising a lens-based optic layout, in accordance with some embodiments. System 800 may comprise one or more alignment features. The one or more alignment features may comprise pegs, détentes, alignment indicators, etc. The one or more pegs may correspond to one or more slots or recesses on the imaging device. The one or more pegs may be configured to interface with the one or more slots or recesses and mechanically adjust a rotation or an orientation of the imaging device. In some embodiments, the alignment system may reduce a likelihood that an imaging device can rotate or move relative to the adaptor once the systems are coupled.

In some cases, system 1000 may comprise an alignment system. The alignment system may comprise a mechanical alignment device 1006. The mechanical alignment device 1006 may move a lens assembly within an optics assembly 350 for directing light to the imaging sensor 340. With the turn of the knob, the lens assembly may translate toward or away from the imaging sensor to adjust a focus of the image on the imaging sensor. System 1000 may comprise an imaging sensor assembly 1004. The image sensor assembly 1004 may comprise and imaging sensor disclosed herein. System 1000 may comprise a camera cable assembly 1002. The camera cable assembly may facilitate coupling of the imaging sensor to a computer system of the present disclosure.

System 1000 may be releasably couplable to an imaging device 1030. Imaging device 1030 may be a C-mount imaging device or a device with an integrated coupler. In the illustrated embodiment, imaging device 1030 may be an imaging device with an integrated coupler.

System 1000 may be releasably couplable to a scope 100. An imaging device may be couplable to interface 1007 of system 1000. Interface 1007 may comprise an example of a connection interface disclosed herein.

In any of the embodiments described herein, the optics or optics assemblies may be configured to diverge light into a plurality of different paths separated by wavelength. In some cases, the optics or optics assemblies may be configured to passively separate wavelengths of light without impacting surgeon view.

### Computer Systems

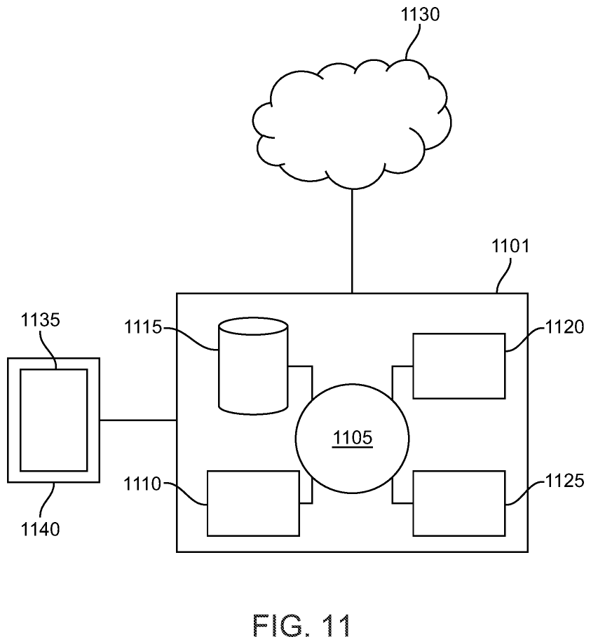

In an aspect, the present disclosure provides computer systems that are programmed or otherwise configured to implement methods of the disclosure, e.g., any of the subject methods for medical imaging. FIG. 11 shows a computer system 1101 that is programmed or otherwise configured to implement a method for medical imaging. The computer system 1101 may be configured to, for example, generate one or more medical images based on the optical signals registered using the imaging sensor and/or the imaging device. The optical signals may be manipulated using one or more optics in order to substantially replicate an image signal received by or from a scope. The computer system 1101 can be an electronic device of a user or a computer system that is remotely located with respect to the electronic device. In some embodiments, the electronic device can be a mobile electronic device.

The computer system 1101 may include a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries, and saved programs. The storage unit 1115 can store user data, e.g., user preferences and user programs. The computer system 1101 in some cases can include one or more additional data storage units that are located external to the computer system 1101 (e.g., on a remote server that is in communication with the computer system 1101 through an intranet or the Internet).

The computer system 1101 can communicate with one or more remote computer systems through the network 1130. For instance, the computer system 1101 can communicate with a remote computer system of a user (e.g., an end user, a medical practitioner, a healthcare worker or provider, an imaging technician, etc.). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine-readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, a portal for a medical practitioner or an imaging technician to view one or more medical images generated using the optical adapter and an imaging device or an imaging sensor coupled to or integrated with the optical adapter. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. For example, the algorithm may be configured to generate one or more medical images based on the optical signals registered using the imaging sensor and/or the imaging device. The optical signals may substantially replicate an image signal received by or from a scope.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A system, comprising:
an optical adapter for visualizing an image of tissue using visible light and non-visible light, wherein the optical adapter is attachable to a scope and an imaging device, and wherein the optical adapter includes,
an optics assembly for directing (i) the visible light to the imaging device and (ii) the non-visible light to an imaging sensor, wherein the optics assembly includes at least one prism, wherein the at least one prism is displaced to compensate for a shift in an optical axis of the visible light caused by one or more components or subcomponents of the optics assembly, and
wherein the visible light exiting the optics assembly toward the imaging device is about the same image size, image collimation, and image orientation as the visible light entering the optics assembly from the scope.

2. The system of claim 1, wherein the imaging device comprises a C-mount imaging device or an imaging device with an integrated coupler.

3. The system of claim 1, wherein the optics assembly comprises a first lens assembly, a second lens assembly, and the at least one prism between the first lens assembly and the second lens assembly.

4. A system, comprising:
an optical adapter for visualizing an image of tissue using visible light and non-visible light, wherein the optical adapter is attachable to a scope and an imaging device, and wherein the optical adapter includes,
an optics assembly for directing (i) the visible light to the imaging device and (ii) the non-visible light to an imaging sensor, wherein the optics assembly includes at least one prism, wherein the at least one prism is displaced to compensate for a shift in an optical axis of the visible light caused by one or more components or subcomponents of the optics assembly,
wherein the imaging device includes a C-mount imaging device or an imaging device with an integrated coupler, and wherein the visible light directed to the imaging device is within a range of about the same image size, image collimation, and image orientation when using either the C-mount imaging device or the imaging device with the integrated coupler.

5. The system of claim 4, wherein the range of the visible light directed to the imaging device is within 20 percent or less different than the visible light entering the optics assembly compared to the visible light exiting the optics assembly toward the imaging device.

6. The system of claim 4, wherein the optics assembly comprises a first lens assembly, a second lens assembly, and the at least one prism between the first lens assembly and the second lens assembly.

10. The system of claim 4, wherein the imaging device comprises the C-mount imaging device and a coupler for the C-mount imaging device.

11. The system of claim 10, wherein the coupler is a native coupler for the C-mount imaging device.

12. The system of claim 1, wherein the at least one prism comprises a porro prism or a roof prism.

13. The system of claim 1, wherein the optics assembly comprises a first optic, a second optic, and third optic, wherein the second optic is positioned between the first optic and the third optic.

14. The system of claim 13, wherein the first optic is configured to direct at least a portion of the visible light to the second optic, and wherein the second optic is configured to manipulate the visible light to adjust a property or a characteristic of the image associated with or derivable from the visible light.

15. The system of claim 14, wherein the third optic is configured to receive the visible light from the second optic and provide substantially parallel beams of the visible light directly to the imaging device, wherein the substantially parallel beams of the visible light substantially replicate an image signal that is received by or from the scope.

17. The system of claim 1, wherein the optics assembly comprises an optical element configured to direct the visible light to the imaging device and the non-visible light to the imaging sensor.

18. The system of claim 17, wherein the optical element comprises a beam splitter.

19. The system of claim 17, wherein the optical element comprises a dichroic mirror or lens.

24. The system of claim 1, wherein the optics assembly comprises at least one achromatic lens to reduce spherical and chromatic aberrations induced or created by the at least one prism.

31. The system of claim 1, wherein the optics assembly is configured to eliminate or prevent a formation, projection, or placement of one or more intermediate image planes on a portion, surface, or edge of the at least one prism.

32. The system of claim 1, wherein the optics assembly is configured to provide an output signal that substantially maintains a quality of an image signal received by or from the scope.

43. The system of claim 1, wherein the optics assembly is configured to actively or passively separate light based on wavelength.

114. A method comprising:
visualizing an image of tissue using visible light and non-visible light with an optical adapter, wherein the optical adapter is attachable to a scope and an imaging device,
directing (i) the visible light to the imaging device and (ii) the non-visible light to an imaging sensor using an optics assembly of the optical adapter, wherein the optics assembly includes at least one prism, and
displacing the at least one prism to compensate for a shift in an optical axis of the visible light caused by one or more components or subcomponents of the optics assembly,
wherein the visible light exiting the optics assembly toward the imaging device is no more than 20 percent different for the same image size, image collimation, and image orientation than the visible light entering the optics assembly from the scope.
